(19) United States
(12) Patent Application Publication
(10) Pub. No.: US 2007/0152569 A1
(43) Pub. Date: Jul. 5, 2007

(54) Novel benzo[b]furan derivatives and their use

(75) Inventors: Jih-Ru Hwu, Hsinchu City (TW); Shwu-Chen Tsay, Taipei (TW); Kao-Shuh Chuang, Hsinchu City (TW)

Correspondence Address: TROXELL LAW OFFICE PLLC, SUITE 1404, 5205 LEESBURG PIKE, FALLS CHURCH, VA, 22041, US

(73) Assignee: Well-being Biochemical Corp.

(21) Appl. No.: 11/324,480

(22) Filed: Jan. 4, 2006

(51) Int. Cl. H01L 51/00; C07D 405/02

(52) U.S. Cl. 313/504; 257/40; 257/E51.02; 549/462

(57) ABSTRACT

The present invention relates to novel benzo[b]furan derivatives, particular to novel benzo[b]furan derivatives having substituents at the 4-position. The present invention also relates to a use of the benzo[b]furan derivatives as electroluminescent material in OLED.

### FIELD OF THE INVENTION

The present invention relates to novel benzo[b]furan derivatives, particular to novel benzo[b]furan derivatives having substituents at the 4-position. The present invention also relates to a use of the benzo[b]furan derivatives as electroluminescent materials in OLED and relates to an OLED containing the same as a dopant.

### BACKGROUND OF THE INVENTION

Organic electroluminescent (EL) device has been known for about two decades. More recently, the organic EL devices include an organic EL element consisting of extremely thin layers between an anode and a cathode.

New organic light-emitting devices (OLEDs) are in great demand by modem electronic industry. They can be used for the manufacture of flat panel display and portable electronic products. By modification of the molecular structure, organic materials can emit the desired color with low-drive voltage. In contrast, difficulties often arise in fabrication of organic materials as efficient light-emitting device.

Nowadays, compounds with blue luminescence are highly desired because of their wide applicability. The existing organic materials with blue light-emitting capability include styrylarylenes (Hosokawa, C.; Higashi, H.; Nakamura, H.; Kusumoto, T. Appl. Phys. Lett. 1995, 3853˜3855), polyphenyls (Zheng, S.; Shi, J. Chem. Mater. 2001, 13, 4405˜4407), perylenes (Jacob, J.; Sax, S.; Piok, T.; List, E. J. W.; Grimsdale, A. C.; Mullen, K. J. Am. Chem. Soc. 2004, 126, 6987˜6995), benzofurans (Anderson, S.; Taylor, P. N.; Verschoor, G. L. B. Chem. Eur. J. 2004, 10, 518˜527; Conley, S. R. U.S. Pat. No. 0,081,853, 2004), indoles (Lin, T. S. U.S. Pat. No. 0,001,967, 2004), oxadiazoles (Jin, S. H.; Kim, M. Y.; Kim, J. Y.; Lee, K.; Gal, Y. S. J. Am. Chem. Soc. 2004, 126, 2474˜2480), thiophenes (Wu, C. C.; Lin, Y. T.; Wong, K. T.; Chen, R. T.; Chien, Y. Y. Adv. Mater. 2004, 16, 61˜65; Kuwahara, A.; Nakano, K.; Nozaki, K. J. Org. Chem. 2005, 70, 413˜419; Wu, Y.; Li, Y.; Gardner, S.; Ong. B. S. J. Am. Chem. Soc. 2005, 127, 614˜618), etc.

Due to critical requirements on physical and optical properties for fabrication, most of the existing devices leave much room to be improved. Accordingly, the present inventors have conducted an investigation on organic materials with blue light-emitting capability and thus completed this invention.

### SUMMARY OF THE INVENTION

The present invention provides functionalized benzo[b]furan and bis-benzo[b]furan derivatives as competent materials for OLEDs.

The present invention provides benzo[b]furan derivatives having the following formula (I): 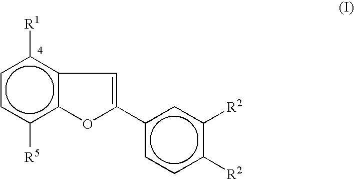
wherein:
R¹ represents a hydrogen, a C₁₋₆ alkyl group, a C₁₋₆ alkoxy group, a C₂₋₇ alkanoyl group, a cyano group, a C₁₋₆ alkylamino group, a C₂₋₇ alkanoylamino group, an aryl group, a heteroaryl group, an arylamino group, a heteroarylamino group or a group of formula −CH═CR³R⁴ in which R³ and R⁴ are the same or different and represent a hydrogen, a C₁₋₆ alkyl group, a C₁₋₆ alkoxy group, a C₂₋₇ alkanoyl group, a C₁₋₆ alkylamino group, a C₂₋₇ alkanoylamino group, an aryl group, a heteroaryl group, an arylamino group, a heteroarylamino group, or a carboxy group;
R²s are the same or different and independently represents a hydrogen, a C₁₋₆ alkyl group, a C₁₋₆ alkoxy group, a C₂₋₇ alkanoyl group, a C₁₋₆ alkylamino group, a C₂₋₇ alkanoylamino group, an aryl group, a heteroaryl group, an arylamino group, or a heteroarylamino group; and
R⁵ represents a hydrogen, a C₁₋₆ alkyl group, a C₁₋₆ alkoxy group, a C₂₋₇ alkanoyl group, a cyano group, a C₁₋₆ alkylamino group, a C₂₋₇ alkanoylamino group, an aryl group, a heteroaryl group, an arylamino group, a heteroarylamino group or a group of formula —CH═CR³R⁴ in which R³ and R⁴ are the same or different and represent a hydrogen, a C₁₋₆ alkyl group, a C₁₋₆ alkoxy group, a C₂₋₇ alkanoyl group, a C₁₋₆ alkylamino group, a C₂₋₇ alkanoylamino group, an aryl group, a heteroaryl group, an arylamino group, a heteroarylamino group, or a carboxy group.
   The present invention also provides bis-benzo[b]furan derivatives having the following formula (II): 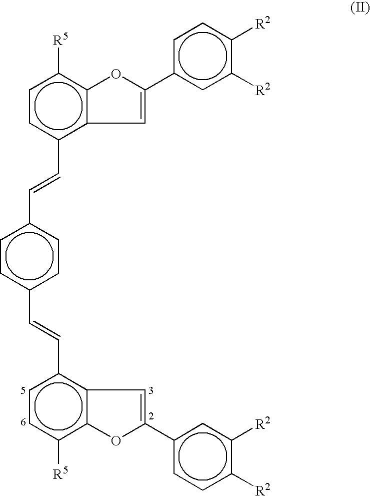
   wherein:
R²s are the same or different and independently represent a hydrogen, a C₁₋₆ alkyl group, a C₁₋₆ alkoxy group, a C₂₋₇ alkanoyl group, a C₁₋₆ alkylamino group, a C₂₋₇ alkanoylamino group, an aryl group, a heteroaryl group, an arylamino group, or a heteroarylamino group; and
R⁵s are the same or different and independently represents a hydrogen, a C₁₋₆ alkyl group, a C₁₋₆ alkoxy group, a C₂₋₇ alkanoyl group, a cyano group, a C₁₋₆ alkylamino group, a C₂₋₇ alkanoylamino group, an aryl group, a heteroaryl group, an arylamino group, a heteroarylamino group or a group of formula —CH═CR³R⁴ in which R³ and R⁴ are the same or different and represent a hydrogen, a C₁₋₆ alkyl group, a C₁₋₆ alkoxy group, a C₂₋₇ alkanoyl group, a C₁₋₆ alkylamino group, a C₂₋₇ alkanoylamino group, an aryl group, a heteroaryl group, an arylamino group, a heteroarylamino group, or a carboxy group.

The present invention also relates to an organic light emitting diode device containing a light emitting layer comprising a host and a blue light emitting dopant, wherein the dopant comprises the benzo[b]furan derivatives of formula (I), the bis-benzo[b]furan derivatives of formula (II), or a mixture thereof.

According to the present organic light emitting diode device, the light emitting layer contains the dopant in an amount of 0.05 to 10.0 wt %, preferably from 0.1 to 6.0 wt % of the host.

According to the present organic light emitting diode device, the dopant can also comprise other light-emitting dopant to achieve different colors. The examples of the other light-emitting dopant include styrylarylenes, polyphenyls, perylenes, benzofurans, indoles, oxadiazoles, thiophenes, etc.

### BRIEF DESCRIPTION OF THE DRAWING

FIG. 1 is shown an ORTEP (Oak Ridge Thermal-Ellipsoid Plot) diagram of diphenylvinylbenzo[b]furan 5c obtained from Example 3.

### DETAILED DESCRIPTION OF THE PRESENT INVENTION

The term “C₁₋₆ alkyl” used herein refers to straight or branched alkyl group having 1-6 carbon atoms, such as methyl, ethyl, n-propyl, i-propyl, n-butyl, i-butyl, t-butyl, n-pentyl, i-pentyl, neopentyl, n-hexyl, i-hexyl, neohexyl, etc.

The term “C₁₋₆ alkoxy” used herein refers to straight or branched alkoxy group having 1-6 carbon atoms, such as methoxy, ethoxy, n-propoxy, i-propoxy, n-butoxy, i-butoxy, t-butoxy, n-pentoxy, i-pentoxy, neopentoxy, n-hexyloxy, i-hexyloxy, neohexyloxy, etc.

The term “C₂₋₇ alkanoyl” used herein refers to a group of formula CO—C₁₋₆ alkyl group in which the C₁₋₆ alkyl group is defined as above.

The term “C₁₋₆ alkylamino” used herein refers to a group of formula —NH—C₁₋₆ alkyl group in which the C₁₋₆ alkyl group is defined as above.

The term “C₂₋₇ alkanoylamino” used herein refers to a group of formula —NHCO—C₁₋₆ alkyl group in which the C₁₋₆ alkyl group is defined as above.

The term “aryl” used herein refers to C₆-C₁₀ aryl group, for example, a phenyl group or a naphthyl group.

The term “heteroaryl” used herein refers to 5-10 member aromatic group containing 1 to 3 heteroatoms selected from nitrogen, oxygen, and sulfur atom.

The term “arylamino” used herein refers to a group of formula —NH-aryl group in which the aryl group is defined as above.

The term “heteroarylamino” used herein refers to a group of formula —NH-heteroaryl group in which the heteroaryl group is defined as above.

The benzo[b]furan derivatives of the formula (I) in which R¹ represents a formyl group, or a cyano group can be prepared by reacting 4-formyl (or 4-cyano)-2-hydroxy-3-iodoanisole 1a (or 1b) with either phenylacetylene 2a or 2b in the presence of (PPh₃)₂PdCl₂, CuI, and triethylamine in a solvent such as dimethylformamide (DMF) at a temperature of from 80° C. to 120° C., preferably at 110° C. to give 4-formyl (or 4-cyano) benzo[b]furan derivatives 3a, 3b (or 3c), as shown in Scheme 1. For the conditions, please refer to (a) McGarry, D. G.; Regan, J. R.; Volz, F. A.; Hulme, C.; Moriarty, K. J.; Djuric, S. W.; Souness, J. E.; Miller, B. E.; Travis, J. J.; Sweeney, D. M. Bioorg. Med. Chem. 1999, 7, 1131˜1139; (b) Flynn, B. L.; Hamel, E.; Jung, M. K. J. Med. Chem. 2002, 45, 2670˜2673.

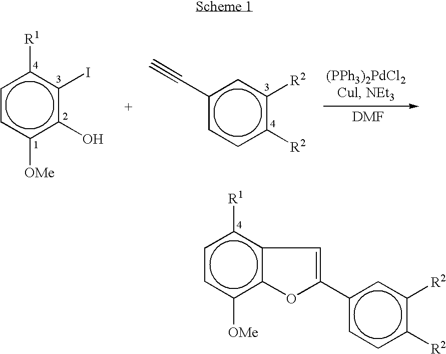

To obtain the benzo[b]furan derivatives of the formula (I) in which R¹ represents a group of formula —CH═CR³R⁴ wherein R³ and R⁴ are the same or different and represent a hydrogen or an aryl group, R⁵ represents a methoxy group, and R²s are the same or different and each represents a hydrogen or an alkoxy group, the 4-formyl benzo[b]furan derivatives 3a and 3b are further allowed to react with benzylphosphonate in the presence of NaH in THF to give vinylbenzo[b]furan derivatives 5a-5d. Alternatively, the 4-formyl benzo[b]furan derivative 3b is further treated with manolic acid in the presence of pyridine and piperidine. Through the Knoevenagel reaction, vinylbenzo[b]furan derivative 5e, i.e. benzo[b]furan derivatives (I) were obtained, in which R¹ represents a group of —CH═CR³R⁴ wherein R³ and R⁴ are the same or different and represent a hydrogen or carboxyl group, R⁵ represents a methoxy group, and R²s are the same or different and each represents a hydrogen or alkoxy group, as shown in Scheme 2. For the conditions, please refer to (a) McGarry, D. G.; Regan, J. R.; Volz, F. A.; Hulme, C.; Moriarty, K. J.; Djuric S. W.; Souness, J. E.; Miller, B. E.; Travis, J. J.; Sweeney, D. M. Bioorg. Med. Chem. 1999, 7, 1131˜1139; (b) Flynn, B. L.; Hamel, E.; Jung, M. K. J. Med. Chem. 2002, 45, 2670˜2673.

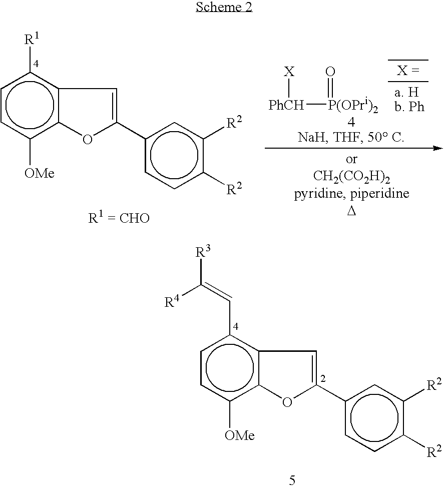

The bis-benzo[b]furan derivatives of the formula (II) can be prepared by reaction of the benzo[b]furan derivatives 3a and 3b with bisphosphonate 6 in the presence of NaH. in a solvent such as THF to give bis-benzo[b]furan derivatives 7a and 7b, as shown in Scheme 3.

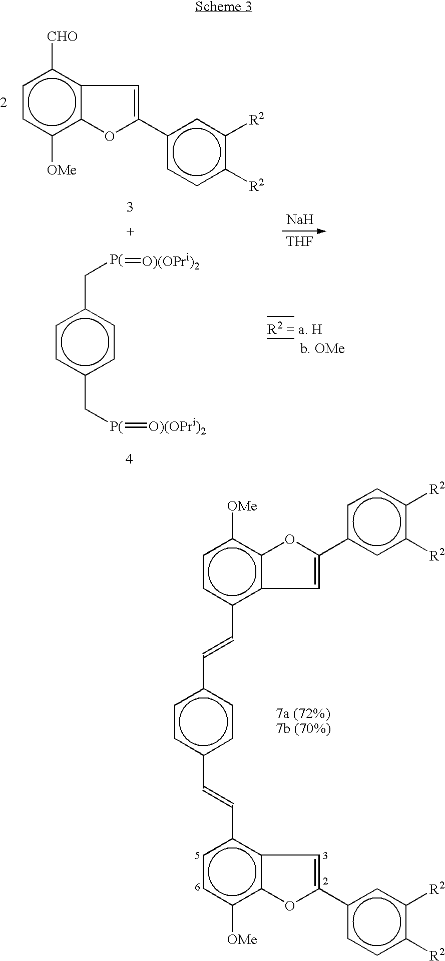

The present invention will now be illustrated by the following examples, which are only provided for illustration purpose but not intended to limit the scope of the present invention.

### EXAMPLE 1

Preparation of Functionalized Benzo[b]Furans (I), in which R¹ Represents a Formyl Group, R⁵ Represents a Methoxy Group, and both R²s Represent a Hydrogen or a Methoxy Group

4-Formyl-2-hydroxy-3-iodoanisole 1a (see: e.g., Markovich, K. M.; Tantishaiyakul, V.; Hamada, A.; Miller, D. D.; Romstedt, K. J.; Shams, G.; Shin, Y.; Fraundorfer, P. F.; Doyle, K.; Feller, D. R. J. Med. Chem. 1992, 35, 466˜479) (1.0 equiv.) was treated with a phenylacetylene (see, e.g., Pelter, A.; Ward, R. S.; Little, G. J. Chem. Soc., Perkin Trans. 1 1990, 10, 2775˜2790) (2.0 equiv.) in the presence of (PPh₃)₂PdCl₂ (0.060 equiv.), and triethylamine (3.0 equiv.) in DMF at 110° C., referred to above-mentioned Scheme 1. The desired 4-formylbenzo[b]furans 3a and 3b (i.e. the benzo[b]furans (I), in which R¹ represents a formyl group, R⁵ represents a methoxy group, and both R² represents a hydrogen or a methoxy group) were generated as solids in 60-63% yields, as shown in

TABLE 1

| Compound | R¹ | R² | R⁵ | Yield |
|---|---|---|---|---|
| 3a | CHO | H | OMe | 63% |
| 3b | CHO | OMe | OMe | 60% |

### EXAMPLE 2

Preparation of Functionalized Benzo[b]Furans (I), in which R¹ Represents a Cyano Group, R⁵ Represents a Methoxy Group, and both R²s Represent a Methoxy Group

4-Cyano-2-hydroxy-3-iodoanisole 1b (1.0 equiv.) was treated with a (3,4-dimethoxyphenyl)acetylene (see, e.g., Pelter, A.; Ward, R. S.; Little, G. J. Chem. Soc., Perkin Trans. 1 1990, 10, 2775˜2790) (2.0 equiv.) in the presence of (PPh₃)₂PdCl₂ (0.060 equiv.) and triethylamine (3.0 equiv.) in DMF at 110° C., referred to above-mentioned Scheme 1. The desired 4-cyanobenzo[b]furans 3c (i.e., the benzo[b]furans (I), in which R¹ represents a cyano group, R⁵ represents a methoxy group, and both R²s represent a methoxy group) were generated as solid in 50% yield, as shown in Table 2.

TABLE 2

| Compound | R¹ | R₂ | R⁵ | Yield |
|---|---|---|---|---|
| 3c | CN | OMe | OMe | 50% |

### EXAMPLE 3

Preparation of Functionalized Benzo[b]Furans (I), in which R¹ Represents a Group of —CH═CR³R⁴ wherein R³ and R⁴ are the same or Different and Represent a Hydrogen, a Phenyl Group or Carboxylic Group, R⁵ Represents a Methoxy Group, and R² Represents a Methoxy Group

The 4-formylbenzo[b]furans 3a and 3b were treated with benzylphosphonate 4a or 4b (see: e.g., Salomon, C. J.; Breuer, E. Tetrahedron Lett. 1995, 36, 6759˜6760) in the presence of NaH in THF to give vinylbenzo[b]furans 5a˜5d in 51-80% yields (i.e., benzo[b]furan derivatives (I) in which R¹ represents a group of —CH═CR³R⁴ wherein R³ and R⁴ are the same or different and represent a hydrogen or a phenyl group, R⁵ represents a methoxy group, and both R²s represents a hydrogen or an alkoxyl group, referred to the above-mentioned Scheme 2).

The 4-formyl benzo[b]furan derivatives 3a and 3b were treated with manolic acid in the presence of pyridine and piperidine through Knoevenagel reaction, to generate vinylbenzo[b]furan derivatives 5e in 70% yield (i.e., benzo[b]furan derivatives (I) in which R¹ represents a group of —CH═CR³R⁴ wherein R³ and R⁴ are the same or different and represent a hydrogen or carboxyl group, R⁵ represents a methoxy group, and R² represents an alkoxyl group, as shown in Table 3.

TABLE 3

| Compound | R² | R³ | R⁴ | R⁵ | Yield |
|---|---|---|---|---|---|
| 5a | H | Ph | H | OMe | 80% |
| 5b | OMe | Ph | H | OMe | 77% |
| 5c | H | Ph | Ph | OMe | 60% |
| 5d | OMe | Ph | Ph | OMe | 51% |
| 5e | OMe | COOH | H | OMe | 70% |

### EXAMPLE 4

Preparation of Bis-benzo[b]Furan Derivatives of the Formula (II), in which all R²s Represent a Hydrogen or a Methoxy Group and both R⁵s Represents a Methoxy Group.

The 4-formylbenzo[b]furans 3a and 3b were treated with bisphosphonate 6 in the presence of NaH in THF, to give the bis-benzu[b]furan derivative 7a and 7b, in which all R²s represent a hydrogen or methoxy group and both R⁵s represents a methoxy group, in 70-72% yields, as shown in Table 4.

TABLE 4

| Compound | R² | R⁵ | Yield |
|---|---|---|---|
| 7a | H | OMe | 72% |
| 7b | OMe | OMe | 70% |

Structures of the synthesized compounds 3b, 3c, 5b, 5c, 5d, 5e, 7a, and 7b of the present invention were fully characterized by ¹H NMR, ¹³C NMR, IR, UV, photoluminescence (PL), and mass spectroscopic methods. For example, bis-benzo[b]furan 7a exhibited one characteristic singlet at 6.85 ppm in its ¹H NMR spectrum for C3-protons. Two doublets with J=8.4 Hz appeared at 7.40 and 7.91 ppm for the C5- and C6-protons; another two doublets with J=16.2 Hz appeared at 7.18 and 7.42 ppm for the vinylic protons. In its ¹³C NMR spectrum, the resonance occurred at 157.09 and 125.70 ppm for the C2- and C3-carbons, respectively, at 128.34 and 122.26 ppm for the two vinylic carbons. In its IR spectrum, one medium absorption band appeared at 1514 cm⁻¹ for the OC═C stretching vibration in the furan moiety.

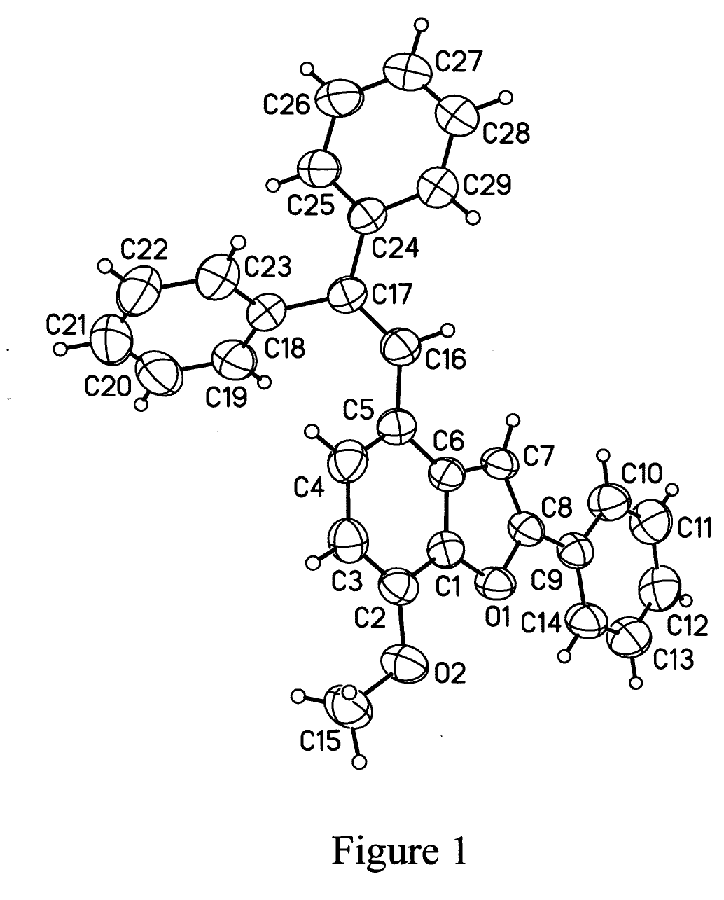

The molecular framework of the compound 5c, which was obtained in single crystal form, was confirmed by using single crystal X-ray diffraction analysis, as shown in FIG. 1. Its triclinic crystal possessed space group P1 with a=11.0613(2) Å, b=11.2060(2) Å, c=18.367(3) Å, α=103.796(3)°, β=98.545(3)°, and γ=90.565(3)°. The dihedral angle was 12° between the benzofuran nucleus and the C2-benzene moiety.

Among mono-benzo[b]furans 3a-3c and 5a-5e, cyano-containing benzo[b]furan derivative 3c exhibited hypsochromic shift and appearing photoluminescence (PL) quantum yield (Φ_{PL}, entry 3 in Table 5). It is due to conjugation of the aromatic π-system therein to an electron-withdrawing cyano group. Thus the cyano group can be used to tune the electronic and the optical properties of benzo[b]furans. On the other hand, bis-benzo[b]furans 7a and 7b showed significant bathochromic shift (entries 9 and 10 in Table 5) with high Φ_{PL} values (0.43-0.46).

Thermal properties of benzo[b]furans were analyzed by differential scanning calorimetry (DSC) and thermal gravimetric analysis (TGA) methods. The results indicate that the bis-benzo[b]furans were more stable than mono-benzo[b]furans. The glass transition temperature (T_{g}), melting temperature, and onset decomposition temperature of bis-benzo[b]furan 7a were 83° C., 282-283° C., and 320° C., respectively. The T_{g} value of bis-benzo[b]furan 7a (83° C.) is higher than the general organic materials used for OLEDs (≧60° C.). While being made as an OLED, bis-benzo[b]furan 7a was found stable at 3.6, 7.0, 11.0, and 15.5 V. Nevertheless, degradation in this OLED appeared in the form of a decrease in device photoluminescence after the voltage reached 17.0 V.

TABLE 5
UV and Photoluminescent Properties of Benzo[b]furan
Derivatives

| | | | UV | PL | PL |
|---|---|---|---|---|---|
| entry | Compound | substituent | λₘₐₓ nm (ε) | λₘₐₓ nm | Φ_{PL}^{a} |
| 1 | 3a | CHO | 332 (13800) | 406 | 0.03 |
| | | | 263 (17310) | | |
| 2 | 3b | CHO | 352 (19640) | 457 | 0.36 |
| | | | 282 (19310) | | |
| 3 | 3c | CN | 330 (25530) | 393 | 0.45 |
| | | | 272 (10020) | | |
| 4 | 5a | CH═CHPh | 314 (26200) | 426 | 0.12 |
| | | | 282 (23200) | | |
| 5 | 5b | CH═CHPh | 342 (29880) | 425 | 0.23 |
| | | | 310 (25240) | | |
| 6 | 5c | CH═CPh₂ | 336 (38360) | 450 | 0.01 |
| | | | 294 (35120) | | |
| 7 | 5d | CH═CPh₂ | 338 (36320) | 462 | 0.01 |
| | | | 300 (12600) | | |
| 8 | 5e | CH═CHCOOH | 342 (8970) | 483 | 0.21 |
| | | | 276 (15630) | | |
| 9 | 7a | C₆H₄CH═CH | 398 (63030) | 463 | 0.43 |
| | | | 296 (41220) | | |
| 10 | 7b | C₆H₄CH═CH | 400 (62020) | 470 | 0.46 |
| | | | 320 (42610) | | |

^{a}The photoluminescence quantum yields in CH₂Cl₂ were measured in comparison with anthracene in ethanol (0.27). The excitation wavelength was fixed at 340 nm.

The HOMO energy of the present benzo[b]furan derivatives was measured with high Φ_{PL} by cyclic voltammetry (CV) with ferrocene (4.8 eV) as the reference. The LUMO energy was calculated from the HOMO and the lowest energy absorption edge of the UV/Visible spectra. Their first oxidation potential (Eₒₓ), HOMO energy, LUMO energy, and band gaps are shown in Table 6. The energy gaps between HOMO and LUMO were significantly lower for bis-benzo[b]furans (entries 4 and 5). This could be attributed to the degrees of conjugation of the aromatic π-system.

TABLE 6
Electrochemical Data of Mono- and Bis-benzo[b]furan
Derivatives

| | | | Eₒₓ (V) | | | |
|---|---|---|---|---|---|---|
| | | | vs | | | Energy |
| | benzo[b] | | Ag/ | HOMO | LUMO | gaps |
| entry | furans | Substituent | AgCl | (eV) | (eV) | (eV) |
| 1 | 3b | CHO | 1.50 | 5.90 | 2.76 | 3.14 |
| 2 | 3c | CN | 1.56 | 5.72 | 2.37 | 3.35 |
| 3 | 5b | CH═CHPh | 1.17 | 5.37 | 2.36 | 3.01 |
| 4 | 7a | C₆H₄CH═CH | 0.98 | 5.26 | 2.48 | 2.78 |
| 5 | 7b | C₆H₄CH═CH | 0.89 | 5.06 | 2.32 | 2.74 |

### PREPARATION EXAMPLE

An Organic Light-emitting Device was Dade by ITO/NPB (40 nm)/Bis-benzo[b]furan 7a (4.0%) in ADN (30 nm)/TPBI (10 nm)/Alq₃ (30 nm)/Mg—Ag (50 nm)/Ag (10 nm). The ADN functioned as a host material layer and the TPBI as the hole blocking material. The abbreviations have the following meanings: ITO=indium tin oxide; NPB=4,4′-bis[N-(1 -naphthyl)-N-phenylamino]biphenyl; ADN=9,10-di(2-naphthyl)anthracene; TPBI=1,3,5-tris(N-phenylbenzimidizol-2-yl)benzene, Alq₃ =tris(8-hydroxyquinoline)aluminum; and Mg—Ag=magnesium-silver alloy (˜10:1). The NPB and Alq₃ were used as the hole and electron transporting material, respectively.

The device of 7a had a turn-on voltage of 3.6 V; its brightness reached 303 cd/m² at 1.0 mA and 6.0 V as well as 1298 cd/m² at 2.48 mA and 7.0 V. The initial color of the device was blue-green (x=0.15 and y=0.25) in the Commission Internationale de L'Eclairage (CIE) chromaticity coordinates. The observed maximum brightness was 53430 cd/m² at 85.5 mA and 15.5 V. The external maximum quantum and power efficiency for the device were 3.75% and 2.03 lm/W, which were achieved at 11V (314 mA/cm², 22418 cd/m²). The device showed the greatest brightness and the maximum power efficiency in comparison with the family of benzofurans disclosed Anderson, S.; Taylor, P. N.; Verschoor, G. L. B. Chem. Eur J. 2004, 10, 518-527; Conley, S. R. U.S. Pat. No. 0,081,853, 2004.

The above experimental data indicates that the benzo[b]furan derivatives of the present invention are useful as electroluminescent materials for emitting blue light, especially those benzo[b]furan derivatives possess functional groups, such as, CHO, CN, CH═CHPh, CH═CPh₂, and CH═CHCOOH group at C4-position.

While the present invention has been described with reference to the specific embodiments thereof, it should be understood by those skilled in the art that various changes and modifications may be made and equivalents may be substituted without departing from the true spirit and scope of the invention. All such changes and modifications are intended to be within in the scope of the claims appended hereto.

What is claimed is:

1. A benzo[b]furan derivative having the following formula (I): 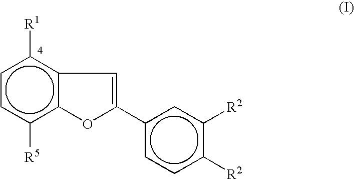
wherein:
R¹ represents a hydrogen, a C₁₋₆ alkyl group, a C₁₋₆ alkoxy group, a C₂₋₇ alkanoyl group, a cyano group, a C₁₋₆ alkylamino group, a C₂₋₇ alkanoylamino group, an aryl group, a heteroaryl group, an arylamino group, a heteroarylamino group or a group of formula —CH═CR³R⁴ in which R³ and R⁴ are the same or different and represent a hydrogen, a C₁₋₆ alkyl group, a C₁₋₆ alkoxyl group, a C₂₋₇ alkanoyl group, a C₁₋₆ alkylamino group, a C₂₋₇ alkanoylarmino group, an aryl group, a heteroaryl group, an arylamino group, a heteroarylamino group, or a carboxyl group,
R²s are the same or different and independently represent a hydrogen, a C₁₋₆ alkyl group, a C₁₋₆ alkoxyl group, a C₂₋₇ alkanoyl group, a C₁₋₆ alkylamino group, a C₂₋₇ alkanoylamino group, an aryl group, a heteroaryl group, an arylamino group, a heteroarylamino group; and
R⁵ represents a hydrogen, a C₁₋₆ alkyl group, a C₁₋₆ alkoxy group, a C₂₋₇ alkanoyl group, a cyano group, a C₁₋₆ alkylamino group, a C₂₋₇ alkanoylamino group, an aryl group, a heteroaryl group, an arylamino group, a heteroarylamino group or a group of formula —CH═CR³R⁴ in which R³ and R⁴ are the same or different and represent a hydrogen, a C₁₋₆ alkyl group, a C₁₋₆ alkoxy group, a C₂₋₇ alkanoyl group, a C₁₋₆ alkylamino group, a C₂₋₇ alkanoylamino group, an aryl group, a heteroaryl group, an arylamino group, a heteroarylamino group, or a carboxy group.

2. The benzo[b]furan derivative according to claim 1, wherein R¹ is a formyl group, both R²s are a hydrogen, and R⁵ is a C₁₋₆ alkoxy group.

3. The benzo[b]furan derivative according to claim 1, wherein R¹ is a formyl group, both R²s are a C₁₋₆ alkoxy group, and R⁵ is a C₁₋₆ alkoxy group.

4. The benzo[b]furan derivative according to claim 3, wherein both R²s are a methoxy group and R⁵ is a methoxy group.

5. The benzo[b]furan derivative according to claim 1, wherein R₁ is a cyano group, both R²s are a C₁₋₆ alkoxyl group, and R⁵ is a C₁₋₆ alkoxy group.

6. The benzo[b]furan derivative according to claim 5, wherein both R²s are a methoxy group and R⁵ is a methoxy group.

7. The benzo[b]furan derivative according to claim 1, wherein R¹ is a group of formula —CH═CR³R⁴ in which R³ and R⁴ are the same or different and represent a hydrogen, a phenyl group, or carboxyl group.

8. The benzo[b]furan derivative according to claim 1, which is used as light emitting materials.

9. A bis-benzo[b]furan derivative having the following formula (II): 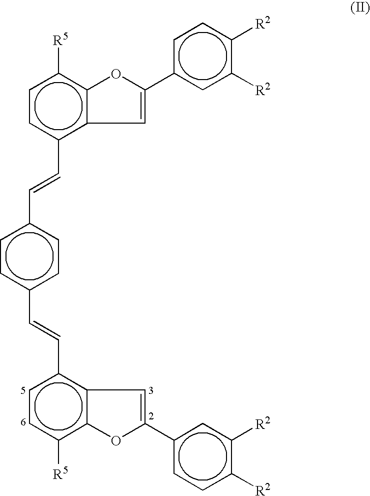
wherein:
R²s are the same or different and independently represent a hydrogen, a C₁₋₆ alkyl group, a C₁₋₆ alkoxy group, a C₂₋₇ alkanoyl group, a C₁₋₆ alkylamino group, a C₂₋₇ alkanoylamino group, an aryl group, a heteroaryl group, an arylamino group, or a heteroarylamino group; and
R⁵s are the same or different and independently represents a hydrogen, a C₁₋₆ alkyl group, a C₁₋₆ alkoxy group, a C₂₋₇ alkanoyl group, a cyano group, a C₁₋₆ alkylamino group, a C₂₋₇ alkanoylamino group, an aryl group, a heteroaryl group, an arylamino group, a heteroarylamino group or a group of formula —CH═CR³R⁴ in which R³ and R⁴ are the same or different and represent a hydrogen, a C₁₋₆ alkyl group, a C₁₋₆ alkoxy group, a C₂₋₇ alkanoyl group, a C₁₋₆ alkylamino group, a C₂₋₇ alkanoylamino group, an aryl group, a heteroaryl group, an arylamino group, a heteroarylamino group, or a carboxy group.

10. The bis-benzo[b]furan derivative according to claim 9, wherein both R²s are a hydrogen and both R⁵s are a methoxy group.

11. The bis-benzo[b]furan derivative according to claim 9, wherein both R²s are a methoxy group and both R⁵s are a methoxy group.

12. The bis-benzo[b]furan derivative according to claim 9, which is used as light emitting materials.

13. An organic light emitting diode device containing a light emitting layer comprising a host and a blue light emitting dopant, wherein the dopant comprises the benzo[b]furan derivatives of formula (I) as claimed in claim 1, the bis-benzo[b]furan derivatives of formula (II) as claimed in claim 9, or a mixture thereof.

14. The organic light emitting diode device according to claim 13, wherein the light emitting layer contains the dopant in an amount of 0.1 to 10.0 wt % of the host.

15. The organic light emitting diode device according to claim 13, which further comprising other light-emitting dopant selected from the group consisting of styrylarylenes, polyphenyls, perylenes, benzofurans, indoles, oxadiazoles, and thiophenes.
